(19) United States
(12) Patent Application Publication
(10) Pub. No.: US 2024/0158361 A1
(43) Pub. Date: May 16, 2024

(54) PROCESS FOR PREPARING AN OLEFIN OXIDE

(71) Applicants: BASF SE, Ludwigshafen am Rhein (DE); Dow Global Technologies LLC, Midland, MI (US)

(72) Inventors: Dominic RIEDEL, Ludwigshafen am Rhein (DE); Dylan SEGERS, Antwerp (BE); Joaquim Henriqu TELES, Ludwigshafen am Rhein (DE); Philip KAMPE, Ludwigshafen am Rhein (DE); Meinolf WEIDENBACH, Stade (DE); Franciscus Johannes Robertus VAN NEER, Hoek (NL); Malte SCHULZ, Stade (DE)

(21) Appl. No.: 18/280,742

(22) PCT Filed: Mar. 10, 2022
(86) PCT No.: PCT/EP2022/056165

§ 371 Date: Sep. 7, 2023

(30) Foreign Application Priority Data
Mar. 11, 2021 (EP) 21162059.6

(51) Int. Cl. C07D 301/12; B01J 8/06

(52) U.S. Cl. CPC C07D 301/12; B01J 8/06

(57) ABSTRACT

The present invention relates to a process for preparing an olefin oxide from a reaction mixture stream in an epoxidation reactor R, wherein R contains z active reaction tubes T(i) arranged in parallel, z≥2, i=1 . . . z, wherein each T(i) comprises a reaction zone Z(i) comprising a heterogeneous epoxidation catalyst, said reaction mixture stream comprising x components C(j), x≥3, j=1 . . . x, the process comprising (i) providing m educt streams E(k), m≥1, k=1 . . . m, wherein each E(k) exhibits a mass flow rate F_{E}(k) and comprises y components C(j), y=1 . . . x, wherein a given component C(j) is contained in at least one E(k); (ii) dividing each E(k) into n educt substreams S(k,i), n≤z, each S(k,i) exhibiting a mass flow rate Fₛ(k,i), wherein to at least one E(k), the inequality (1) applies: Formulas (1), (2), (3), (iii) providing n reaction mixtures streams M(i) comprising the x components C(j), said providing comprising, for each i, either combining and admixing the n educt substreams S(k,i) obtaining the n reaction mixtures M(i) if m>1, or passing on the n educt substreams S(k,i) as the n reaction mixtures M(i) if m=1; (iv) feeding each M(i) obtained according to (iii) into Z(i) and contacting each M(i) in Z(i) with the epoxidation catalyst under epoxidation reaction conditions; wherein the x components C(j) comprise hydrogen peroxide, an organic solvent, and the olefin. The present invention further relates to an olefin oxide obtained or obtainable from said process.

The present invention relates to a process for preparing an olefin oxide from a reaction mixture stream in an epoxidation reactor R, wherein R contains z active reaction tubes T(i) arranged in parallel, z≥2, i=1 . . . z, wherein each T(i) comprises a reaction zone Z(i) comprising a heterogeneous epoxidation catalyst, said reaction mixture stream comprising x components C(j), x≥3, j=1 . . . x. The present invention further relates to an olefin oxide, obtained or obtainable from said process.

Fixed bed reactors for catalyzed reactions are well known in the art (see, for example, W. Ruppel in Ullmann's Encyclopedia of Industrial Chemistry, “Catalytic Fixed bed Reactors”, Wiley-VCH Verlag GmbH & Co. KGaA, Jul. 15, 2012) and include tubular as well as multitubular reactors. The reactors and thus the tube(s) can be arranged horizontally or vertically, the reactors can be operated in batch or continuous mode, the reactants are fed to the reactors in liquid and/or gaseous form as individual or combined streams. For example, a multitubular reactor, i.e. a reactor where the catalyst is present in fixed bed form of solid catalyst material within each tube of a plurality of tubes, can be arranged in a horizontal manner with several parallel tubes and the feed stream comprising the educts and optionally solvent can be fed to said reactor as one single liquid phase. Another example is a trickle bed reactor with a plurality of parallel tubes which are arranged vertically. The trickle bed reactor is a three-phase catalytic reactor in which liquid and gas phases flow concurrently up-/downward through a fixed bed of solid catalyst particles, which are present within the tubes. Trickle bed reactors are disclosed, for example, by Ranade et al. (Trickle Bed Reactors: Reactor Engineering and Applications, Vivek V. Ranade, Raghunath Chaudhari, Prashant R. Gunjal, Elsevier, Mar. 18, 2011).

Even if fixed bed reactors are known for decades, there is still a need to improve the conversion of the educts and thus also the achievable overall yield of the desired product(s). One prominent example where this need is present is in the synthesis of olefin oxides (epoxides) and most prominently of propylene oxide, which is one of the most important chemical (intermediate) products in industry. It represents the starting compound for a broad spectrum of products, such as polyurethane, propylene glycol, solvents, surfactants or de-icing agents. For example, for a multitubular reactor used in the preparation of propylene oxide from propene and (aqueous) hydrogen peroxide in a solvent (a so called “HPPO process”) over an epoxidation catalyst comprising a zeolitic material, problems arise if there is a distribution in hydrogen peroxide (H₂O₂) conversion over the tubes or if higher reactor temperatures are required to achieve a sufficient H₂O₂ conversion, both resulting in a reduced yield with respect to propylene oxide and in increasing amounts of unwanted by-products such as 1-methoxypropan-2-ol, 2-methoxypropan-1-ol and propylene glycol dimethyl ether. The problems increase over the operation time of the reactor due to, for example, salt precipitation or back-flushing, or in general due to the long operation times.

It was therefore an object of the present invention to provide a process for the preparation of an olefin oxide, which is effective and allows to achieve a favorable yield with respect to the olefin oxide.

The invention thus relates to a process for preparing an olefin oxide from a reaction mixture stream in an epoxidation reactor R, wherein R contains z active reaction tubes T(i) arranged in parallel, z≥2, i=1 . . . z, wherein each T(i) comprises a reaction zone Z(i) comprising a heterogeneous epoxidation catalyst, said reaction mixture stream comprising x components C(j), x≥3, j=1 . . . x, the process comprising
(i) providing m educt streams E(k), m≥1, k=1 . . . m, wherein each E(k) exhibits a mass flow rate F_{E}(k) and comprises y components C(j), y=1 . . . x, wherein a given component C(j) is contained in at least one E(k);
(ii) dividing each E(k) into n educt substreams S(k,i), n≤z, each S(k,i) exhibiting a mass flow rate F_{S}(k,i), wherein to at least one E(k), the inequality (1) applies:

$${σ}^{n} ( k ) = \frac{σ ( {F}_{E} ( k ) )}{{F}_{E}^{n} ( k )} \leq 0.4 \qquad ( 1 )$$

$$σ ( {F}_{E} ( k ) ) = \sqrt{\frac{{\sum }_{i = 1}^{n} {( {F}_{S} ( k , i ) - {F}_{E}^{n} ( k ) )}^{2}}{n}} \qquad ( 2 )$$

$${F}_{E}^{n} = \frac{{F}_{E} ( k )}{n} \qquad ( 3 )$$

(iii) providing n reaction mixtures streams M(i) comprising the x components C(j), said providing comprising, for each i, either combining and admixing the n educt substreams S(k,i) obtaining the n reaction mixtures M(i) if m>1, or passing on the n educt substreams S(k,i) as the n reaction mixtures M(i) if m=1;
(iv) feeding each M(i) obtained according to (iii) into Z(i) and contacting each M(i) in Z(i) with the epoxidation catalyst under epoxidation reaction conditions;
   wherein the x components C(j) comprise hydrogen peroxide, an organic solvent, and the olefin.

Surprisingly, it was found that, especially when each reaction zone Z(i) is filled with heterogeneous epoxidation catalyst, maintaining σⁿ(k) below or equal to 0.4 allows to minimize the amount of unwanted by-products.

In the process for preparing an olefin oxide according to the present invention, it is preferred that inequality (1) applies to each E(k). Preferably, m>1 and E(1) comprises two components C(1) and C(2) and is essentially free of C(3), and E(2) comprises one component C(3) and is essentially free of C(1) and C(2).

In the process for preparing an olefin oxide according to the present invention, it is preferred that C(1) is hydrogen peroxide, C(2) is organic solvent, and C(3) is the olefin. Preferably, x≥4 and the x components C(j) further comprise water.

In the process for preparing an olefin oxide according to the present invention, it is preferred that m>1 and E(1) comprises three components C(1), C(2) and C(4) and is essentially free of C(3), and E(2) comprises one component C(3) and is essentially free of C(1), C(2) and C(4).

In the process for preparing an olefin oxide according to the present invention, it is preferred that C(1) is hydrogen peroxide, C(2) is organic solvent, C(3) is the olefin, and C(4) is water.

In the process for preparing an olefin oxide according to the present invention, it is preferred that σⁿ(k) is in the range of from 0 to 0.4, more preferred in the range of from 0 to 0.039, more preferred in the range of from 0 to 0.035, more preferred in the range from 0 to 0.03.

In the process for preparing an olefin oxide according to the present invention, it is preferred that m is 1, 2, or 3, preferably 1 or 2.

In the process for preparing an olefin oxide according to the present invention, it is preferred that at least one, more preferred all educt streams E(k) are liquid streams.

In the process for preparing an olefin oxide according to the present invention, it is preferred that m=1, the process comprising
(i′) providing an educt stream E exhibiting a mass flow rate F_{E} and comprising y components C(j), y=1 . . . x;
(ii′) dividing E into n educt substreams S(i), n≤z, each S(i) exhibiting a mass flow rate F_{S}(i), wherein the inequality (1) applies:

$${σ}^{n} ( k ) = \frac{σ ( {F}_{E} ( k ) )}{{F}_{E}^{n} ( k )} \leq 0.4 \qquad ( 1 )$$

$$σ ( {F}_{E} ( k ) ) = \sqrt{\frac{{\sum }_{i = 1}^{n} {( {F}_{S} ( k , i ) - {F}_{E}^{n} ( k ) )}^{2}}{n}} \qquad ( 2 )$$

$${F}_{E}^{n} = \frac{{F}_{E} ( k )}{n} \qquad ( 3 )$$

(iii′) feeding each M(i) into Z(i) and contacting each M(i) in Z(i) with the epoxidation catalyst under epoxidation reaction conditions.

In this embodiment, S(i) is preferably identical to M(i), in other words feeding each M(i) into Z(i) and contacting each M(i) in Z(i) with the epoxidation catalyst under epoxidation reaction conditions is here equal to feeding each S(i) into Z(i) and contacting each S(i) in Z(i) with the epoxidation catalyst under epoxidation reaction conditions.

In the process for preparing an olefin oxide according to the present invention, it is preferred that C(1) is hydrogen peroxide, C(2) is organic solvent, and C(3) is water.

In the process for preparing an olefin oxide according to the present invention, it is preferred that x≥4 and C(1) is hydrogen peroxide, C(2) is organic solvent, C(3) is water, and C(4) is olefin.

In the process for preparing an olefin oxide according to the present invention, it is preferred that on is in the range of from 0 to 0.4, more preferred in the range of from 0 to 0.039, more preferred in the range of from Oto 0.035, more preferred in the range of from 0 to 0.03.

In the process for preparing an olefin oxide according to the present invention, it is preferred that z is at least 100, more preferred at least 1,000, more preferred in the range of from 1,000 to 100,000, more preferred in the range of from 10,000 to 50,000.

In the process for preparing an olefin oxide according to the present invention, it is preferred that—more preferred if x≥4 and C(1) is hydrogen peroxide, C(2) is organic solvent, C(3) is water, and C(4) is olefin—0.9z<n≤z, more preferred 0.95z<n≤z, more preferred 0.98z<n≤z, more preferred 0.98z<n≤z.

In the process for preparing an olefin oxide according to the present invention, it is preferred that the volume of the filling of a tube of the multitubular reactor with heterogeneous epoxidation catalyst deviates from the filled volume of each other tube by less than 10%, more preferred in the range from 0 to 10%.

In the process for preparing an olefin oxide according to the present invention, it is preferred that the contacting of each M(i) in Z(i) with the epoxidation catalyst under epoxidation reaction conditions in (iv) is carried out at an absolute pressure in the reaction zones in the range of from 0.5 to 5.0 MPa, more preferred in the range of from 1.5 to 3.0 MPa, more preferred in the range of from 1.8 to 2.8 MPa.

In the process for preparing an olefin oxide according to the present invention, it is preferred that the contacting of each M(i) in Z(i) with the epoxidation catalyst under epoxidation reaction conditions in (iv) is carried out at a temperature in the reaction zones in the range of from 25 to 75° C., more preferred in the range of from 28 to 70° C., more preferred in the range of from 30 to 65° C. The temperature in the reaction zone(s) Z(i) in the context of this application is defined as the entrance temperature of the cooling medium to the mantle of the reactor. In case there is more than one entrance or even more than one reaction zone each with a separate entrance for the cooling medium, then the temperature in the reaction zone will be defined as the weight averaged temperature of all the cooling medium feeding streams.

In the process for preparing an olefin oxide according to the present invention, it is preferred that the weight ratio of C(3) olefin:C(1) hydrogen peroxide (w/w) in the educt stream E is in the range of from 1:1 to 5:1, more preferred in the range of from 1:1 to 2:1 or in the range of from 3:1 to 5:1.

In the process for preparing an olefin oxide according to the present invention, it is preferred that the weight ratio of C(2) organic solvent:C(1) hydrogen peroxide (w/w) in the educt stream E is in the range of from 15:1 to 5:1, more preferred in the range of from 12:1 to 6:1, more preferred in the range of from 12:1 to 9:1 or in the range of from 8:1 to 6:1.

In the process for preparing an olefin oxide according to the present invention, it is preferred that the weight ratio of C(2) organic solvent:C(3) olefin (w/w) in the educt stream E is in the range of from 10:1 to 1:0.1, preferably in the range of from 9:1 to 1:1, more preferred in the range of from 9:1 to 7:1 or in the range of from 1.5:1 to 1:1.

In the process for preparing an olefin oxide according to the present invention, it is preferred that in the epoxidation reaction conditions according to (iv) comprise trickle bed conditions or fixed bed conditions. Generally, no specific restrictions exist regarding the conditions under which the contacting in the reaction zone(s) Z(i) with the heterogenous epoxidation catalyst takes place provided that an efficient epoxidation of C(3) olefin takes place. Preferably, the epoxidation reaction conditions according to (iv) comprise trickle bed conditions or fixed bed conditions, wherein fixed bed conditions are more preferred. Preferably, these conditions are applied in a reactor wherein the catalyst is present in a fixed bed. “Trickle bed conditions” preferably mean that the reaction is preferably carried out at temperatures and pressures at which the reaction mixture is present partly in a liquid phase and partly in a gaseous phase, with the catalyst being present in a fixed bed. In embodiments with fixed bed conditions, the reaction is preferably carried out at temperatures and pressures at which the reaction mixture is liquid and no gas phase is present in the reaction zone, wherein two or more liquid phases may exist, with the catalyst being present in a fixed bed.

In the process for preparing an olefin oxide according to the present invention, it is preferred that the heterogeneous epoxidation catalyst comprises a zeolitic material having a framework structure comprising Si, O, and Ti. Preferably, the zeolitic material comprises Ti in an amount in the range of from 0.2 to 5 weight-%, more preferred in the range of from 0.5 to 4 weight-%, more preferred in the range of from 1.0 to 3 weight-%, more preferred in the range of from 1.2 to 2.5 weight-%, more preferred in the range of from 1.4 to 2.2 weight-%, calculated as elemental Ti and based on the total weight of the zeolitic material. Preferably, the zeolitic material having a framework structure comprising Si, O, and Ti comprised in the epoxidation catalyst is a titanium zeolite having ABW, ACO, AEI, AEL, AEN, AET, AFG, AFI, AFN, AFO, AFR, AFS, AFT, AFX, AFY, AHT, ANA, APC, APD, AST, ASV, ATN, ATO, ATS, ATT, ATV, AWO, AWW, BCT, BEA, BEC, BIK, BOG, BPH, BRE, CAN, CAS, CDO, CFI, CGF, CGS, CHA, CHI, CLO, CON, CZP, DAC, DDR, DFO, DFT, DOH, DON, EAB, EDI, EMT, EPI, ERI, ESV, ETR, EUO, FAU, FER, FRA, GIS, GIU, GME, GON, GOO, HEU, IFR, ISV, ITE, ITH, ITQ, ITW, IWR, IWW, JBW, KFI, LAU, LEV, LIO, LOS, LOV, LTA, LTL, LTN, MAR, MAZ, MCM-22(S), MCM-36, MCM-56, MEI, MEL, MEP, MER, MIT-1, MMFI, MFS, MON, MOR, MSE, MSO, MTF, MTN, MTT, MTW, MWW, NAB, NAT, NEES, NON, NPO, OBW, OFF, OSI, OSO, PAR, PAU, PHI, PON, RHO, RON, RRO, RSN, RTE, RTH, RUT, RWR, RWY, SAO, SAS, SAT, SAV, SBE, SBS, SBT, SFE, SFF, SFG, SFH, SFN SFO, SGT, SOD, SSY, STF, STI, STT, TER, THO, TON, TSC, UEI, UFI, UOZ, USI, UTL, VET, VFI, VNI, VSV, WEI, WEN, YUG, ZON SVR, SVY framework structure or a mixed structure of two or more of these framework types; more preferred the zeolitic material having a framework structure comprising Si, O, and Ti is a titanium zeolite having an MFI framework type, an MEL framework type, an MWW framework type, an MCM-22(S) framework type, an MCM-56 framework type, an IEZ-MWW framework type, an MCM-36 framework type, an ITQ framework type, a BEA framework type, a MOR framework type, or a mixed structure of two or more of these framework types; more preferred an MFI framework type, or an MWW framework type; more preferred the zeolitic material having a framework structure comprising Si, O, and Ti has framework type MFI; more preferred the zeolitic material having a framework structure comprising Si, O, and Ti is a titanium silicalite-1 (TS-1). Framework types such as MCM-22(S), MCM-56, IEZ-MWW, ITQ (delaminated MWW), MIT-1, and MCM-36 are titanium zeolites having framework structures related to MWW framework structure, obtained or obtainable therefrom or from the respective two dimensional precursor by, for example, layer expansion and/or post-modification. In a fresh TS-1, i.e. a TS-1 which has not already been used as catalyst, preferably from 95 to 100 weight-%, more preferred from 98 to 100 weight-%, more preferred from 99 to 100 weight-%, more preferred from 99.5 to 100 weight-%, more preferred from 99.9 to 100 weight-% of the zeolitic material consist of Si, O, Ti and optionally H. In case the zeolitic material having a framework structure comprising Si, O, and Ti comprised in the epoxidation catalyst is a titanium zeolite having an MWW framework type, said titanium zeolite of framework type MWW is also referred to as “TiMWW”, which relates to a zeolite of framework structure MWW which contains titanium as isomorphous substitution element in the zeolitic framework. Preferably, the zeolitic framework is essentially free of aluminum and essentially consists of silicon, titanium, and oxygen. Preferably, at least 99 weight-%, more preferred at least 99.5 weight-%, more preferred at least 99.9 weight-% of the zeolitic framework consist of silicon, titanium, and oxygen. Optionally, the titanium zeolite of framework structure type MWW may comprise extra-framework titanium which is to be understood as every titanium species which is not part of the MWW zeolitic framework. In addition to the titanium, the titanium zeolite of framework structure type MWW may comprise at least one further element other than titanium, silicon, and oxygen. Generally, it is conceivable that this at least one further element is an isomorphous substitution element which is part of the MWW zeolitic framework structure. Preferably, this at least one further element is not an isomorphous substitution element. Such a further element which is not an isomorphous substitution element can be applied to the zeolite by, for example, a spray process, a wet impregnation process such as an incipient wetness process, or any other suitable process. Preferably, the at least one further element is selected from the group consisting of Al, B, Zr, V, Nb, Ta, Cr, Mo, W, Mn, Fe, Co, Ni, Zn, Ga, Ge, In, Sn, Pb, Pd, Pt, Au, Cd and a combination of two or more, preferably from the group consisting of B, Zr, V, Nb, Ta, Cr, Mo, W, Mn, Fe, Co, Ni, Zn, Ga, Ge, In, Sn, Pb, Pd, Pt, Au, Cd combination of two or more. More preferred, the titanium zeolite of framework structure type MWW contains zinc as further element in addition to titanium, silicon, and oxygen. More preferred, the titanium zeolite of framework structure type MWW contains zinc as the sole further element in addition to titanium, silicon, and oxygen. More preferred, the titanium zeolite of framework structure type MWW contains zinc as the sole further element in addition to titanium, silicon, and oxygen wherein at least 99 weight-%, more preferred at least 99.5 weight-%, more preferred at least 99.9 weight-% of the zeolitic framework structure consist of silicon, titanium, and oxygen. More preferred, in case the titanium zeolite of framework structure type MWW contains zinc as the sole further element, at least 99 weight-%, more preferred at least 99.5 weight-%, more preferred at least 99.9 weight-% of the titanium zeolite of framework structure type MWW consist of zinc, titanium, silicon, and oxygen; this titanium zeolite of framework structure type MWW which contains zinc as the sole further element is also referred to as “ZnTiMWW”.

In the process for preparing an olefin oxide according to the present invention, it is preferred that the heterogeneous epoxidation catalyst further comprises a binder. Preferably, the heterogeneous epoxidation catalyst is in the form of a molding, more preferred in the form of an extrudate or a granule. Preferably, from 95 to 100 weight-%, more preferred from 98 to 100 weight-%, more preferred from 99 to 100 weight-%, more preferred from 99.5 to 100 weight-%, more preferred from 99.9 to 100 weight-% of the molding consist of the zeolitic material and the binder. Preferably, from 95 to 100 weight-%, more preferred from 98 to 100 weight-%, more preferred from 99 to 100 weight-%, more preferred from 99.5 to 100 weight-%, more preferred from 99.9 to 100 weight-% of the binder comprised in the molding consist of Si and O.

In the process for preparing an olefin oxide according to the present invention, it is preferred that the heterogeneous epoxidation catalyst, more preferred the molding, comprises the binder, calculated as SiO₂, in an amount in the range of from 2 to 90 weight-%, preferably in the range of from 5 to 70 weight-%, more preferred in the range of from 10 to 50 weight-%, more preferred in the range of from 15 to 30 weight-%, more preferred in the range of from 20 to 25 weight-%, based on the total weight of the epoxidation catalyst, preferably based on the total weight of the molding and/or wherein the heterogeneous epoxidation catalyst, preferably the molding, comprises the zeolitic material in an amount in the range of from 10 to 98 weight-%, preferably in the range of from 30 to 95 weight-%, more preferred in the in the range of from 50 to 90 weight-%, more preferred in the range of from 70 to 85 weight-%, more preferred in the range of from 75 to 80 weight-%, based on the total weight of the heterogeneous epoxidation catalyst, preferably based on the total weight of the molding.

Generally, the contacting of M(i) in Z(i) with the epoxidation catalyst according to (iv) can be carried out in any appropriate way. Thus, for example, it can be carried out in a batch mode or in at least one semi-continuously operated mode or in continuous mode. The continuous mode of operation is preferred. Preferably, at least (iv) is carried out continuously, wherein more preferred at least (iii) and (iv), more preferred (ii), (iii) and (iv), more preferred (i), (ii), (iii) and (iv), are carried out continuously.

In the process for preparing an olefin oxide according to the present invention, it is preferred that the hydrogen peroxide is provided as aqueous hydrogen peroxide solution, which preferably has a total organic carbon content (TOC) in the range of from 100 to 800 mg per kg hydrogen peroxide comprised in the aqueous hydrogen peroxide solution, preferably in the range of from 120 to 750 mg per kg hydrogen peroxide comprised in the aqueous hydrogen peroxide solution, more preferred in the range of from 150 to 700 mg per kg hydrogen peroxide comprised in the aqueous hydrogen peroxide solution, determined according to DIN EN 1484. Preferably, the hydrogen peroxide has a pH in the range of from 0 to 3.0, more preferred in the range of from 0.1 to 2.5, more preferred in the range of from 0.5 to 2.3, determined with a pH sensitive glass electrode according to CEFIC PEROXYGENS H₂O₂ AM-7160 standard (2003). The pH is to be understood as being determined using a pH sensitive glass electrode wherein the liquid aqueous system is in an inert atmosphere which avoids, for example, that the liquid aqueous system comes into contact with atmospheric carbon dioxide which, if absorbed in the liquid aqueous system, would reduce the pH. Preferably, the hydrogen peroxide comprises from 20 to 85 weight-%, more preferred from 30 to 75 weight-%, more preferred from 40 to 70 weight-% of hydrogen peroxide relative to the total weight of the aqueous hydrogen peroxide solution. Preferably, the hydrogen peroxide is obtained or obtainable from an anthraquinone process.

According to an embodiment of the present invention, it is preferred to employ an aqueous hydrogen peroxide solution, which is obtained as crude hydrogen peroxide solution by extraction of a mixture which results from a process known as anthraquinone process (see, e.g., Ullmann's Encyclopedia of Industrial Chemistry, 5^{th} edition, volume A 13 (1989) pages 443-466), wherein a solution of an anthraquinone is used containing an alkyl group preferably having from 2 to 10 carbon atoms, more preferred a 2-6 carbon atoms, more preferred 2, 5 or 6 carbon atoms, and where the solvent used usually consists of a mixture of at least two different solvents. Preferably, mixtures of two solvents or mixtures of three solvents are used. Preferably, none of the solvents used in the anthraquinone process is a nitrogen containing substance. This solution of the anthraquinone is usually referred to as the working solution. In this process, the hydrogen peroxide formed in the course of the anthraquinone process is generally separated by extraction from the respective working solution after a hydrogenation/re-oxidation cycle. Said extraction can be performed preferably with essentially pure water, and the crude aqueous hydrogen peroxide solution is obtained. It is generally possible to further purify and/or concentrate the thus obtained crude aqueous hydrogen peroxide solution by distillation. It is possible to use crude aqueous hydrogen peroxide solution which has not been subjected to purification and/or concentration by distillation and it is also possible to use an aqueous hydrogen peroxide solution which has been subjected to purification and/or concentration by distillation. Further, it is generally possible to subject the crude aqueous hydrogen peroxide solution to a further extraction stage wherein a suitable extracting agent, preferably an organic solvent is used. More preferred, the organic solvent used for this further extraction stage is the same solvent which is used in the anthraquinone process. Preferably the extraction is performed using just one of the solvents in the working solution and most preferably using just the most nonpolar solvent of the working solution. In case the crude aqueous hydrogen peroxide solution is subjected to such further extraction stage, a so-called crude washed hydrogen peroxide solution is obtained. According to a preferred embodiment of the present invention, the crude washed hydrogen peroxide solution is used as the aqueous hydrogen peroxide solution. The production of a crude solution is described, for example, in European patent application EP 1 122 249 A1. As to the term “essentially pure water”, reference is made to paragraph 10, page 3 of EP 1 122 249 A1 which is incorporated by reference. The hydrogen peroxide can also be treated to remove trace metals, for example, as described in the WO 2015/049327 A1 before use.

It is conceivable that the hydrogen peroxide is prepared in situ in the reaction zone from hydrogen and oxygen, preferably in the presence of a suitable noble metal catalyst comprised in the reaction zone according to (ii). A suitable noble metal catalyst preferably comprises one or more of palladium, platinum, silver, gold, rhodium, iridium, ruthenium and osmium. Preferably, the noble metal catalyst comprises palladium. The noble metal catalyst is preferably supported on a carrier, wherein the carrier preferably comprises one or more of SiO₂, Al₂O₃, B₂O₃, GeO₂, Ga₂O₃, ZrO₂, TiO₂, MgO, carbon and one or more zeolites, preferably one or more titanium zeolites. More preferred, the carrier comprises the epoxidation catalyst comprising a titanium zeolite. If hydrogen peroxide is prepared in the reaction zone according to (ii) in situ from hydrogen and oxygen, the reaction mixture provided in (i) comprises propene, hydrogen, oxygen, water, and organic solvent.

In the process for preparing an olefin oxide according to the present invention, it is preferred that the organic solvent is an organic epoxidation solvent, more preferred the organic solvent is selected from the group consisting of alcohol, acetonitrile, propionitrile and mixtures of two or more thereof; more preferred selected from the group consisting of alcohol, acetonitrile and mixtures of alcohol and acetonitrile; more preferred the organic solvent comprises at least an alcohol, wherein the alcohol is preferably a C₁ to C₅ mono alcohol or a mixture of two or more C₁ to C₅ alcohols, more preferred the alcohol comprises at least methanol. According to a preferred embodiment, the organic solvent is methanol.

In the process for preparing an olefin oxide according to the present invention, it is preferred that the olefin is propene. Generally, it is conceivable to use a pure or essentially pure propene as starting material. Preferably, a mixture of propene and propane is used. More preferred a technical propene grade according to an international norm like for instance ASTM D5273 or DIN 51622 is used. If a mixture of propene and propane is used, the weight ratio of propene propane is preferably at least 7:3. For example, commercially available propene can be employed which may be either a polymer grade propene or a chemical grade propene. Typically, polymer grade propene has a propene content in the range of from 99 to 99.8 weight-% and a propane content in the range of from 0.2 to 1 weight-%. Chemical grade propene typically has a propene content in the range of from 92 to 98 weight-% and a propane content in the range of from 2 to 8 weight-%. According to a preferred embodiment of the present invention, a mixture of propene and propane is used which has a propene content in the range of from 99 to 99.8 weight-% and a propane content in the range of from 0.2 to 1 weight-%.

No restrictions exist regarding the water used for the reaction mixture. It is conceivable to use, for example, water which is treated with NH₃ but water not having been treated with NH₃ can also be used. Preferably deionized water is used for the reaction mixture. The deionized water can be obtained using ion-exchangers of using condensate. Typical grades of deionized water are defined in ISO 3696 of 1987 and all grades described there can be used within the scope of this invention. The water may additionally contain traces of corrosion inhibiting additives like ammonia, hydrazine or hydroxylamine in which case it should have a pH value in the range of 7 to 9 (determined with a pH sensitive glass electrode according to CEFIC PEROXYGENS H₂O₂ AM-7160 standard (2003)). Preferably, the water used does not contain corrosion inhibiting additives.]

In the process for preparing an olefin oxide according to the present invention, it is preferred that x≥4 and the x components C(j) further comprise an additive, preferably selected from the group consisting of potassium salt, ammonia, ammonium salt, etidronic acid, salt of etidronic acid, and mixtures of two or more thereof.

The term “potassium salt” comprises
potassium salt of inorganic acid, preferably selected from the group consisting of potassium halide (KCl, KBr), potassium nitrate, potassium sulfate, potassium hydrogen sulfate, potassium hydroxide, potassium perchlorate, potassium cyanate, dipotassium carbonate, potassium hydrogen carbonate, and mixtures of two or more thereof;
potassium salt of phosphorus oxyacid, preferably selected from potassium dihydrogen phosphate, dipotassium hydrogen phosphate, potassium phosphate, monobasic potassium pyrophosphate, dibasic potassium pyrophosphate, tribasic potassium pyrophosphate, tetrabasic potassium pyrophosphate, and mixtures of two or more thereof;
potassium oxide, preferably selected from the group consisting of potassium oxide (K₂O), potassium superoxide (KO₂), potassium peroxide (K₂O₂), and mixtures of two or more thereof;
potassium salt of organic acid, preferably selected from the group consisting of potassium salt of monocarboxylic acid, preferably having from 1 to 6, more preferred from 1 to 5, more preferred from 1 to 4, more preferred from 1 to 3 carbon atoms, more preferred selected from the group consisting of formic acid, acetic acid, propionic acid, and mixtures of two or more thereof; dicarboxylic acid, preferably having from 2 to 6, more preferred from 2 to 4 carbon atoms, more preferred selected from the group consisting of oxalic acid, malonic acid, succinic acid, tartaric acid, and mixtures of two or more thereof; tricarboxylic acid, preferably having from 6 to 10 carbon atoms, more preferred selected from the group consisting of citric acid, isocitric acid, propane-1,2,3-tricarboxylic acid, and mixtures of two or more thereof; and tetracarboxylic acid; and mixtures of two or more thereof.

Preferably, the potassium salt is selected from the group consisting of potassium dihydrogen phosphate, dipotassium hydrogen phosphate, potassium formate, potassium acetate, dipotassium carbonate, potassium hydrogen carbonate, and mixtures of two or more thereof, more preferred from the group consisting of potassium dihydrogen phosphate, dipotassium hydrogen phosphate, potassium formate, potassium acetate, potassium hydrogen carbonate, and mixtures of two or more thereof.

The term “ammonium salt” preferably refers to ammonium salt of phosphorus oxyacid, more preferred selected from the group consisting of ammonium dihydrogen phosphate, diammonium hydrogen phosphate, ammonium phosphate, monobasic ammonium pyrophosphate, dibasic ammonium pyrophosphate, tribasic ammonium pyrophosphate, tetrabasic ammonium pyrophosphate, and mixtures of two or more thereof.

Etidronic acid is (1-hydroxy-1-phosphonoethyl)phosphonic acid. The term “salt of etidronic acid” preferably refers to potassium salt of etidronic acid, ammonium salt of etidronic acid and mixtures of two or more thereof, more preferred selected from the group consisting of monobasic potassium etidronate, dibasic potassium etidronate, tribasic potassium etidronate, tetrabasic potassium etidronate, potassium monobasic ammonium etidronate, dibasic ammonium etidronate, tribasic ammonium etidronate, tetrabasic ammonium etidronate, and mixtures of two or more thereof.

In the process for preparing an olefin oxide according to the present invention, it is preferred that the additive is selected from the group consisting of potassium dihydrogen phosphate, dipotassium hydrogen phosphate, potassium formate, potassium acetate, potassium hydrogen carbonate, etidronic acid, ammonium dihydrogen phosphate, diammonium hydrogen phosphate, ammonia and mixtures of two or more thereof, preferably form the group consisting pf potassium dihydrogen phosphate, dipotassium hydrogen phosphate, etidronic acid, ammonia and mixtures of two or more thereof.

The present invention further relates to an olefin oxide, preferably propylene oxide, obtained or obtainable from the process as described above.

The present invention is further illustrated by the following set of embodiments and combinations of embodiments resulting from the dependencies and back-references as indicated. In particular, it is noted that in each instance where a range of embodiments is mentioned, for example in the context of a term such as “any one of embodiments (1) to (4)”, every embodiment in this range is meant to be explicitly disclosed for the skilled person, i.e. the wording of this term is to be understood by the skilled person as being synonymous to “any one of embodiments (1), (2), (3), and (4)”. Further, it is explicitly noted that the following set of embodiments is not the set of claims determining the extent of protection, but represents a suitably structured part of the description directed to general and preferred aspects of the present invention.
1. A process for preparing an olefin oxide from a reaction mixture stream in an epoxidation reactor R, wherein R contains z active reaction tubes T(i) arranged in parallel, z≥2, i=1 . . . z, wherein each T(i) comprises a reaction zone Z(i) comprising a heterogeneous epoxidation catalyst, said reaction mixture stream comprising x components C(j), x≥3, j=1 . . . x, the process comprising
   (i) providing m educt streams E(k), m≥1, k=1 . . . m, wherein each E(k) exhibits a mass flow rate F_{E}(k) and comprises y components C(j), y=1 . . . x, wherein a given component C(j) is contained in at least one E(k);
   (ii) dividing each E(k) into n educt substreams S(k,i), n≤z, each S(k,i) exhibiting a mass flow rate F_{S}(k,i), wherein to at least one E(k), the inequality (1) applies:

$${σ}^{n} ( k ) = \frac{σ ( {F}_{E} ( k ) )}{{F}_{E}^{n} ( k )} \leq 0.4 \qquad ( 1 )$$

$$σ ( {F}_{E} ) = \sqrt{\frac{{\sum }_{i = 1}^{n} {( {F}_{S} ( i ) - {F}_{E}^{n} )}^{2}}{n}} \qquad ( 2 )$$

$${F}_{E}^{n} = \frac{{F}_{E}}{n} \qquad ( 3 )$$

(iii) providing n reaction mixtures streams M(i) comprising the x components C(j), said providing comprising, for each i, either combining and admixing the n educt substreams S(k,i) obtaining the n reaction mixtures M(i) if m>1, or passing on the n educt substreams S(k,i) as the n reaction mixtures M(i) if m=1;
(iv) feeding each M(i) obtained according to (iii) into Z(i) and contacting each M(i) in Z(i) with the epoxidation catalyst under epoxidation reaction conditions;
wherein the x components C(j) comprise hydrogen peroxide, an organic solvent, and the olefin.
2. The process of embodiment 1, wherein the inequality (1) applies to each E(k).
   3. The process of embodiment 1 or 2, wherein m>1 and E(1) comprises two components C(1) and C(2) and is essentially free of C(3), and E(2) comprises one component C(3) and is essentially free of C(1) and C(2).
4. The process of embodiment 3, wherein C(1) is hydrogen peroxide, C(2) is organic solvent, and C(3) is the olefin.
5. The process of embodiment 1 or 2, wherein x≥4 and the x components C(j) further comprise water.
6. The process of embodiment 5, wherein m>1 and E(1) comprises three components C(1), C(2) and C(4) and is essentially free of C(3), and E(2) comprises one component C(3) and is essentially free of C(1), C(2) and C(4).
7. The process of embodiment 6, wherein C(1) is hydrogen peroxide, C(2) is organic solvent, C(3) is the olefin, and C(4) is water.
8. The process of any one of embodiments 1 to 7, wherein σⁿ(k) is in the range of from 0 to 0.4, preferably in the range of from 0 to 0.039, more preferably in the range of from 0 to 0.035, more preferred in the range from 0 to 0.03.
9. The process of any one of embodiments 1 to 8, wherein m is 1, 2, or 3, preferably 1 or 2.
10. The process of any one of embodiment 1 to 9, wherein at least one, preferably all educt streams E(k) are liquid streams.
11. The process of any one of embodiments 1 to 10, wherein m=1, the process comprising
   (i′) providing an educt stream E exhibiting a mass flow rate F_{E} and comprising y components C(j), y=1 . . . x;
   (ii′) dividing E into n educt substreams S(i), n≤z, each S(i) exhibiting a mass flow rate F_{S}(i), wherein the inequality (1) applies:

$${σ}^{n} = \frac{σ ( {F}_{E} )}{{F}_{E}^{n}} \leq 0.4 \qquad ( 1 )$$

$$σ ( {F}_{E} ) = \sqrt{\frac{{\sum }_{i = 1}^{n} {( {F}_{S} ( i ) - {F}_{E}^{n} )}^{2}}{n}} \qquad ( 2 )$$

$${F}_{E}^{n} = \frac{{F}_{E}}{n} \qquad ( 3 )$$

(iii′) feeding each M(i) into Z(i) and contacting each M(i) in Z(i) with the epoxidation catalyst under epoxidation reaction conditions.
12. The process of embodiment 11, wherein C(1) is hydrogen peroxide, C(2) is organic solvent, and C(3) is water.
13. The process of embodiment 11, wherein x≥4 and C(1) is hydrogen peroxide, C(2) is organic solvent, C(3) is water, and C(4) is olefin.
14. The process of any one of embodiment 11 to 13, wherein an is in the range of from 0 to 0.4, preferably in the range of from 0 to 0.039, more preferably in the range of from Oto 0.035, more preferred in the range of from 0 to 0.03.
15. The process of any one of embodiments 1 to 14, wherein z is at least 100, preferably at least 1,000, more preferably in the range of from 1,000 to 100,000, more preferably in the range of from 10,000 to 50,000.
16. The process of any one of embodiment 1 to 15, preferably of embodiment 13, wherein 0.9z<n≤z, preferably 0.95z<n≤z, more preferably 0.98z<n≤z, more preferably 0.98z<n≤z.
17. The process of any one of embodiments 1 to 16, wherein the volume of the filling of a tube of the multitubular reactor with heterogeneous epoxidation catalyst deviates from the filled volume of each other tube by less than 10%, preferably in the range from 0 to 10%.
18. The process of any one of embodiments 1 to 17, wherein the contacting of each M(i) in Z(i) with the epoxidation catalyst under epoxidation reaction conditions in (iv) is carried out at an absolute pressure in the reaction zones in the range of from 0.5 to 5.0 MPa, preferably in the range of from 1.5 to 3.0 MPa, more preferably in the range of from 1.8 to 2.8 MPa.
19. The process of any one of embodiments 1 to 18, wherein the contacting of each M(i) in Z(i) with the epoxidation catalyst under epoxidation reaction conditions in (iv) is carried out at a temperature in the reaction zones in the range of from 25 to 75° C., preferably in the range of from 28 to 70° C., more preferably in the range of from 30 to 65° C.
20. The process of any one of embodiments 13 to 19, wherein the weight ratio of C(3) olefin:C(1) hydrogen peroxide (w/w) in the educt stream E is in the range of from 1:1 to 5:1, preferably in the range of from 1:1 to 2:1 or in the range of from 3:1 to 5:1.
21. The process of any one of embodiments 13 to 20, wherein the weight ratio of C(2) organic solvent:C(1) hydrogen peroxide (w/w) in the educt stream E is in the range of from 15:1 to 5:1, preferably in the range of from 12:1 to 6:1, more preferably in the range of from 12:1 to 9:1 or in the range of from 8:1 to 6:1.
22. The process of any one of embodiments 13 to 21, wherein the weight ratio of C(2) organic solvent:C(3) olefin (w/w) in the educt stream E is in the range of from 10:1 to 1:0.1, preferably in the range of from 9:1 to 1:1, more preferably in the range of from 9:1 to 7:1 or in the range of from 1.5:1 to 1:1.
23. The process of any one of embodiments 1 to 22, wherein the epoxidation reaction conditions according to (iv) comprise trickle bed conditions.
24. The process of any one of embodiments 1 to 22, wherein the epoxidation reaction conditions according to (iv) comprise fixed bed conditions.
25. The process of any one of embodiments 1 to 24, wherein the heterogeneous epoxidation catalyst comprises a zeolitic material having a framework structure comprising Si, O, and Ti.
26. The process of embodiment 25, wherein the zeolitic material comprises Ti in an amount in the range of from 0.2 to 5 weight-%, preferably in the range of from 0.5 to 4 weight-%, more preferably in the range of from 1.0 to 3 weight-%, more preferably in the range of from 1.2 to 2.5 weight-%, more preferably in the range of from 1.4 to 2.2 weight-%, calculated as elemental Ti and based on the total weight of the zeolitic material.
27. The process of embodiment 25 or 26, wherein the zeolitic material having a framework structure comprising Si, O, and Ti comprised in the epoxidation catalyst is a titanium zeolite having ABW, ACO, AEI, AEL, AEN, AET, AFG, AFI, AFN, AFO, AFR, AFS, AFT, AFX, AFY, AHT, ANA, APC, APD, AST, ASV, ATN, ATO, ATS, ATT, ATV, AWO, AWW, BCT, BEA, BEC, BIK, BOG, BPH, BRE, CAN, CAS, CDO, CFI, CGF, CGS, CHA, CHI, CLO, CON, CZP, DAC, DDR, DFO, DFT, DOH, DON, EAB, EDI, EMT, EPI, ERI, ESV, ETR, EUO, FAU, FER, FRA, GIS, GIU, GME, GON, GOO, HEU, IFR, ISV, ITE, ITH, ITQ, ITW, IWR, IWW, JBW, KFI, LAU, LEV, LIO, LOS, LOV, LTA, LTL, LTN, MAR, MAZ, MCM-22(S), MCM-36, MCM-56, MEI, MEL, MEP, MER, MIT-1, MMFI, MFS, MON, MOR, MSE, MSO, MTF, MTN, MTT, MTW, MWW, NAB, NAT, NEES, NON, NPO, OBW, OFF, OSI, OSO, PAR, PAU, PHI, PON, RHO, RON, RRO, RSN, RTE, RTH, RUT, RWR, RWY, SAO, SAS, SAT, SAV, SBE, SBS, SBT, SFE, SFF, SFG, SFH, SFN SFO, SGT, SOD, SSY, STF, STI, STT, TER, THO, TON, TSC, UEI, UFI, UOZ, USI, UTL, VET, VFI, VNI, VSV, WEI, WEN, YUG, ZON SVR, SVY framework structure or a mixed structure of two or more of these framework types; more preferably the zeolitic material having a framework structure comprising Si, O, and Ti is a titanium zeolite having an MFI framework type, an MEL framework type, an MWW framework type, an MCM-22(S) framework type, an MCM-56 framework type, an IEZ-MWW framework type, an MCM-36 framework type, an ITQ framework type, a BEA framework type, a MOR framework type, or a mixed structure of two or more of these framework types; more preferably an MFI framework type, or an MWW framework type; more preferred the zeolitic material having a framework structure comprising Si, O, and Ti has framework type MFI; more preferably the zeolitic material having a framework structure comprising Si, O, and Ti is a titanium silicalite-1 (TS-1).
28. The process of any one of embodiments 25 to 27, wherein the heterogeneous epoxidation catalyst further comprises a binder.
29. The process of embodiment 28, wherein the heterogeneous epoxidation catalyst is in the form of a molding, preferably in the form of an extrudate or a granule.
30. The process of embodiment 28 or 29, wherein from 95 to 100 weight-%, preferably from 98 to 100 weight-%, more preferably from 99 to 100 weight-%, more preferably from 99.5 to 100 weight-%, more preferably from 99.9 to 100 weight-% of the molding consist of the zeolitic material and the binder.
31. The process of any one of embodiments 25 to 30, wherein from 95 to 100 weight-%, preferably from 98 to 100 weight-%, more preferably from 99 to 100 weight-%, more preferably from 99.5 to 100 weight-%, more preferably from 99.9 to 100 weight-% of the binder comprised in the molding consist of Si and O.
32. The process of embodiment 31, wherein the heterogeneous epoxidation catalyst, preferably the molding, comprises the binder, calculated as SiO₂, in an amount in the range of from 2 to 90 weight-%, preferably in the range of from 5 to 70 weight-%, more preferably in the range of from 10 to 50 weight-%, more preferably in the range of from 15 to 30 weight-%, more preferably in the range of from 20 to 25 weight-%, based on the total weight of the epoxidation catalyst, preferably based on the total weight of the molding and/or wherein the heterogeneous epoxidation catalyst, preferably the molding, comprises the zeolitic material in an amount in the range of from 10 to 98 weight-%, preferably in the range of from 30 to 95 weight-%, more preferred in the in the range of from 50 to 90 weight-%, more preferred in the range of from 70 to 85 weight-%, more preferred in the range of from 75 to 80 weight-%, based on the total weight of the heterogeneous epoxidation catalyst, preferably based on the total weight of the molding.
33. The process of any one of embodiments 1 to 32, wherein at least (iv) is carried out continuously, wherein preferably at least (iii) and (iv), more preferred (ii), (iii) and (iv), more preferred (i), (ii), (iii) and (iv), are carried out continuously.
34. The process of any one of embodiments 1 to 33, wherein the hydrogen peroxide is provided as aqueous hydrogen peroxide solution, which preferably has a total organic carbon content (TOC) in the range of from 100 to 800 mg per kg hydrogen peroxide comprised in the aqueous hydrogen peroxide solution, preferably in the range of from 120 to 750 mg per kg hydrogen peroxide comprised in the aqueous hydrogen peroxide solution, more preferably in the range of from 150 to 700 mg per kg hydrogen peroxide comprised in the aqueous hydrogen peroxide solution, determined according to DIN EN 1484.
35. The process of any one of embodiments 1 to 34, wherein the hydrogen peroxide has a pH in the range of from 0 to 3.0, preferably in the range of from 0.1 to 2.5, more preferably in the range of from 0.5 to 2.3, determined with a pH sensitive glass electrode according to AM7160.
36. The process of any one of embodiments 1 to 35, wherein the hydrogen peroxide comprises from 20 to 85 weight-%, preferably from 30 to 75 weight-%, more preferably from 40 to 70 weight-% of hydrogen peroxide relative to the total weight of the aqueous hydrogen peroxide solution.
37. The process of any one of embodiments 1 to 36, wherein the hydrogen peroxide is obtained or obtainable from an anthraquinone process.
38. The process of any one of embodiments 1 to 37, wherein the organic solvent is an organic epoxidation solvent, preferably the organic solvent is selected from the group consisting of alcohol, acetonitrile, propionitrile and mixtures of two or more thereof; more preferred selected from the group consisting of alcohol, acetonitrile and mixtures of alcohol and acetonitrile; more preferred the organic solvent comprises at least an alcohol, wherein the alcohol is preferably a C1 to C5 mono alcohol or a mixture of two or more C1 to C5 alcohols, more preferred the alcohol comprises at least methanol.
39. The process of any one of embodiments 1 to 38, wherein x≥4 and the x components C(j) further comprise an additive, preferably selected from the group consisting of potassium salt, ammonia, ammonium salt, etidronic acid, salt of etidronic acid, and mixtures of two or more thereof.
40. The process of embodiment 39, wherein the additive is selected from the group consisting of potassium dihydrogen phosphate, dipotassium hydrogen phosphate, potassium formate, potassium acetate, potassium hydrogen carbonate, etidronic acid, ammonium dihydrogen phosphate, diammonium hydrogen phosphate, ammonia and mixtures of two or more thereof, preferably form the group consisting pf potassium dihydrogen phosphate, dipotassium hydrogen phosphate, etidronic acid, ammonia and mixtures of two or more thereof.
41. Olefin oxide, preferably propylene oxide, obtained or obtainable from the process of any one of embodiments 1 to 40.

It is explicitly noted that the preceding set of embodiments is not the set of claims determining the extent of protection, but represents a suitably structured part of the description directed to general and preferred aspects of the present invention.

The present invention is further illustrated by the following reference examples, comparative examples, and examples.

### EXAMPLES

### Simulations

All simulations were done with process simulation software Aspen Plus v.11. The components used in the process simulation and their characteristics respectively, were taken from the Dortmund Database. The kinetic model of Russo et al. was used without further modification (V. Russo, R. Tesser, E. Santacesaria, M. Di Serio, Ind. Eng. Chem. Res. 2013, 52, 1168-1178).

### Reference Example 1: Set Up of Multitubular Reactor (Flooded Reactor, Upstream Mode)

A multitubular reactor (the reactor) was used with a bundle of 20,000 vertically arranged tubes made of stainless steel with a length of 2,000 mm of each tube and inner diameter of each tube of 28.5 mm. Through the tubes, the reaction mixture was passed from the bottom to the top, i.e. in upstream mode.

The heat transfer inside the tube was modelled according an axial flow model. The heat transfer outside of the tubes was assumed as non-limiting for the overall heat transport.

The pressure in the reactor was kept constant at 2.5 MPa.

The reactor was further equipped with a cooling jacket. As cooling medium, water was passed through the cooling jacket in upstream mode. The flow rate of the cooling medium was adjusted so that the temperature difference between the inlet temperature and the outlet temperature of the cooling medium was 2° C. at most. Typically, this temperature difference was only about 0.5° C.

All 20,000 tubes T(i) of the reactor each contained (in the reaction zone Z(i)) 620 g of strands of a heterogeneous titanium silicalite-1 (TS-1) catalyst, which was considered an ideal filling. The TS-1 catalyst had a bulk density in the range from 470 to 480 g/l. The titanium content of each TS-1 strand was 0.71 wt.-%, the Si content was 44 wt.-%, each based on the total weight of the TS-1 strand. The pore volume of the strands, determined via Hg porosimetry according to DIN 66133:1993-06, was 73 ml/g. The strands had a diameter of 1.5 mm and the length was in the range from 3 to 5 mm. The tubes had a inner diameter of 40 mm.

The reaction feed, .e. the educt stream E, consisting of methanol (69.0 wt.-%), propene (11.9 wt.-%), water (11.7 wt.-%) and hydrogen peroxide (7.4 wt.-%) consisted of one single liquid phase and was fed to the multitubular reactor with a mass flow rate F_{E}=323 t/h at room temperature (25° C.), divided into 20,000 substreams S(i), each substream S(i) exhibiting a mass flow rate F_{S}(i)=16.15 kg/h, wherein one substream S(i) was fed (as M(i)) to each of the 20,000 tubes. Also, the liquid reaction mixture in the reactor consisted of one single phase. The tubes were assumed to be cooled by an ideal cooling medium of constant temperature and the heat transfer coefficient from the tubes to the cooling medium was assumed to be high enough to not limit the heat transfer. The temperature of the cooling medium was chosen in such a way that the overall conversion of hydrogen peroxide at the exit of the multitubular reactor was exactly 90%

### Example 1: Epoxidation of Propene—Distribution of Pressure Loss in the Tubes According Normal Distribution, σⁿ in the Range from Zero to 0.04

A reaction of propene with hydrogen peroxide in the presence of methanol and water over a TS-1 catalyst resulting in the desired main product propylene oxide and one or more unwanted by-product(s), which are selected from 1-methoxypropan-2-ol, 2-methoxypropan-1-ol and propylene glycol dimethyl ether, according to Reference Example 1 was modelled.

As reference, an idealized reactor with σⁿ=0 (that is, all tubes have exactly identical pressure loss) was taken, wherein the sum of the weights of the unwanted by-products 1-methoxypropan-2-ol, 2-methoxypropan-1-ol and propylene glycol dimethyl ether was set as 100%.

The modelling for a value of σⁿ=0.026 resulted in an increase of the unwanted by-products of +0.5% compared to the reference.

### Comparative Example 1: Epoxidation of Propene—Distribution of Pressure Loss in the Tubes According Normal Distribution, σⁿ>0.04

The modelling of Example 1 was repeated, also based on the assumption of a distribution of pressure loss in the tubes according to a normal distribution. Contrary to Example 1, the modelling was based on a value of σⁿ=0.066. This resulted in an increase of the unwanted by-products of +5.5% compared to the reference.

### Comparative Example 2: Epoxidation of Propene—Distribution of Pressure Loss in Ideally Filled Tubes According Normal Distribution, σⁿ=0

The modelling of Example 1 was repeated. Contrary to Example 1, only 19,999 tubes of the reactor were filled according to Reference Example 1; one (1) tube was not filled with TS-1 catalyst, i.e. remained empty.

For the 19,999 filled tubes of the reactor, a distribution of pressure loss in the tubes was assumed according to a normal distribution. Contrary to Example 1, the modelling was based on a value of σⁿ=0 for these 19,999 tubes. The use of one empty tube resulted in an increase of the unwanted by-products of +30.6% compared to the reference.

### Comparative Example 3: Epoxidation of Propene—Distribution of Pressure Loss in Ideally Filled Tubes According Normal Distribution, σⁿ=0.026

The modelling of Example 1 was repeated. Contrary to Example 1, only 19,800 tubes of the reactor were filled according to Reference Example 1 with TS-1 catalyst; 200 tubes were filled with a catalytically inactive material.

For all 20,000 filled tubes of the reactor, a distribution of pressure loss in the tubes was assumed according to a normal distribution. Contrary to Example 1, the modelling was based on a value of σⁿ=0.026 for all tubes. The use of 1% of the tubes (200 of 20,000 tubes) resulted in an increase of the unwanted by-products of +6.1% compared to the reference.

### Comparative Example 4: Epoxidation of Propene—Distribution of Pressure Loss in Ideally Filled Tubes According Triangular Symmetric Distribution, σⁿ=0.062

The modelling of Example 1 was repeated. Contrary to Example 1, the modelling was based on a triangular symmetric distribution of pressure loss. Contrary to Example 1, the modelling was based on a value of σⁿ=0.062 for all tubes. This resulted in an increase of the unwanted by-products of +10.8% compared to the reference.

Example 1 and Comparative Examples 1 to 4 were also simulated based on a first alternative multitubular reactor with 20,000 tubes operated in flooded, downstream mode) and also based on a second alternative multitubular reactor with 20,000 tubes operated in trickle bed mode with fed from the top. Comparable results as in Example 1 and Comparative Examples 1 to 4 were achieved for the first and second alternative multitubular reactors, which means that the amount of the unwanted by-products was in each case the same value as in the respective Example or Comparative Example ±0.05%.

Summary

The results from Example 1 and from Comparative Examples 1 to 4 are summarized below in table 1, wherein the increase of the overall amount of the unwanted by-products 1-methoxypropan-2-ol, 2-methoxypropan-1-ol and propylene glycol dimethyl ether is reported as %-increase relative to the ideal case, where the overall amount of the unwanted by-products was defined as 100%.

TABLE 1
Results from epoxidation of propene with hydrogen peroxide
according to Example 1 and Comparative Examples 1 to 4.

| | | % increase of | |
|---|---|---|---|
| Example | σⁿ | by-products | Remarks |
| Example 1 | 0.026 | +0.5% ^{a)} | All 20,000 tubes ideally filled with TS-1 catalyst, |
| | | | normal distribution of pressure loss/flow rates |
| Comparative | 0.066 | +5.5% ^{a)} | All 20,000 tubes ideally filled with TS-1 catalyst, |
| Example 1 | | | normal distribution of pressure loss/flow rates |
| Comparative | 0 | +30.6% ^{a)} | 19,999 tubes ideally filled with TS-1 catalyst + 1 |
| Example 2 | | | empty tube, normal distribution of pressure |
| | | | loss/flow rates |
| Comparative | 0.026 | +6.1% ^{a)} | Normal distribution of pressure loss, but 1% of |
| Example 3 | | | the 20,000 tubes (in this case 200 tubes) are |
| | | | filled with catalytically inactive material |
| Comparative | 0.062 | +10.8% ^{b)} | All 20,000 tubes ideally filled with TS-1 catalyst, |
| Example 4 | | | triangular symmetric distribution of pressure |
| | | | loss/flow rates |

^{a)} Compared to the case where σⁿ is zero with all tubes ideally filled with TS-1 catalyst and based on a normal distribution of pressure loss/flow rates.
^{b)} Compared to the case where σⁿ is zero with all tubes ideally filled with TS-1 catalyst and based on a triangular symmetric distribution of pressure loss/flow rates.

It was found that, especially when each tube is filled with heterogeneous epoxidation catalyst (TS-1 catalyst), maintaining an below or equal to 0.4 allowed to minimize the amount of unwanted by-products.

### CITED LITERATURE

W. Ruppel in Ullmann's Encyclopedia of Industrial Chemistry, “Catalytic Fixed bed Reactors”, Wiley-VCH Verlag GmbH & Co. KGaA, Jul. 15, 2012
Trickle Bed Reactors: Reactor Engineering and Applications, Vivek V. Ranade, Raghunath Chaudhari, Prashant R. Gunjal, Elsevier, Mar. 18, 2011
“II—Pressure Drop in Packed Tubes”, Industrial and Engineering Chemistry, pages 913 to 919, August 1931
Ullmann's Encyclopedia of Industrial Chemistry, 5^{th} edition, volume A 13 (1989) pages 443-466
EP1122249A1
WO 2015/049327 A1
V. Russo, R. Tesser, E. Santacesaria, M. Di Serio, Ind. Eng. Chem. Res. 2013, 52, 1168-1178

1.-15. (canceled)

16. A process for preparing an olefin oxide from a reaction mixture stream in an epoxidation reactor R, wherein R contains z active reaction tubes T(i) arranged in parallel, z≥100, i=1 . . . z, wherein each T(i) comprises a reaction zone Z(i) comprising a heterogeneous epoxidation catalyst, said reaction mixture stream comprising x components C(j), x≥3, j=1 . . . x, the process comprising:
(i) providing m educt streams E(k), m≥1, k=1 . . . m, wherein each E(k) exhibits a mass flow rate F_{E}(k) and comprises y components C(j), y=1 . . . x, wherein a given component C(j) is contained in at least one E(k);
(ii) dividing each E(k) into n educt substreams S(k,i), n≤z, each S(k,i) exhibiting a mass flow rate F_{S}(k,i), wherein to at least one E(k), the inequality (1) applies:
$${σ}^{n} ( k ) = \frac{σ ( {F}_{E} ( k ) )}{{F}_{E}^{n} ( k )} \leq 0.4 \qquad ( 1 )$$
$$σ ( {F}_{E} ( k ) ) = \sqrt{\frac{{\sum }_{i = 1}^{n} {( {F}_{S} ( k , i ) - {F}_{E}^{n} ( k ) )}^{2}}{n}} \qquad ( 2 )$$
$${F}_{E}^{n} = \frac{{F}_{E} ( k )}{n} \qquad ( 3 )$$
(iii) providing n reaction mixtures streams M(i) comprising the x components C(j), said providing comprising, for each i, either combining and admixing the n educt substreams S(k,i) obtaining the n reaction mixtures M(i) if m>1, or passing on the n educt substreams S(k,i) as the n reaction mixtures M(i) if m=1;
(iv) feeding each M(i) obtained according to (iii) into Z(i) and contacting each M(i) in Z(i) with the epoxidation catalyst under epoxidation reaction conditions;
wherein the x components C(j) comprise hydrogen peroxide, an organic solvent, and the olefin.

17. The process of claim 16, wherein the inequality (1) applies to each E(k).

18. The process of claim 16, wherein m>1 and E(1) comprises two components C(1) and C(2) and is essentially free of C(3), and E(2) comprises one component C(3) and is essentially free of C(1) and C(2).

19. The process of claim 18, wherein C(1) is hydrogen peroxide, C(2) is organic solvent, and C(3) is the olefin.

20. The process of claim 16, wherein x≥4 and the x components C(j) further comprise water.

21. The process of claim 20, wherein m>1 and E(1) comprises three components C(1), C(2) and C(4) and is essentially free of C(3), and E(2) comprises one component C(3) and is essentially free of C(1), C(2) and C(4).

22. The process of claim 16, wherein σⁿ(k) is in the range of from 0 to 0.4.

23. The process of claim 16, wherein m is 1, 2, or 3.

24. The process of claim 16, wherein m=1, the process comprising:
(i′) providing an educt stream E exhibiting a mass flow rate F_{E} and comprising y components C(j), y=1 . . . x;
(ii′) dividing E into n educt substreams S(i), n≤z, each S(i) exhibiting a mass flow rate F_{S}(i), wherein the inequality (1) applies:
$${σ}^{n} = \frac{σ ( {F}_{E} )}{{F}_{E}^{n}} \leq 0.4 \qquad ( 1 )$$
$$σ ( {F}_{E} ) = \sqrt{\frac{{\sum }_{i = 1}^{n} {( {F}_{S} ( i ) - {F}_{E}^{n} )}^{2}}{n}} \qquad ( 2 )$$
$${F}_{E}^{n} = \frac{{F}_{E}}{n} \qquad ( 3 )$$
(iii′) feeding each M(i) into Z(i) and contacting each M(i) in Z(i) with the epoxidation catalyst under epoxidation reaction conditions.

25. The process of claim 24, wherein C(1) is hydrogen peroxide, C(2) is organic solvent, and C(3) is water; and/or wherein x≥4 and C(1) is hydrogen peroxide, C(2) is organic solvent, C(3) is water, and C(4) is olefin.

26. The process of claim 24, wherein σⁿ is in the range of from 0 to 0.4.

27. The process of claim 16, wherein z is at least 1,000.

28. The process of claim 16, wherein 0.9z<n≤z.

29. The process of claim 16, wherein the volume of the filling of a tube of the multitubular reactor with heterogeneous epoxidation catalyst deviates from the filled volume of each other tube by less than 10%.

30. An olefin oxide obtained from the process of claim 16.
